(19) United States
(12) Patent Application Publication
(10) Pub. No.: US 2009/0192571 A1
(43) Pub. Date: Jul. 30, 2009

(54) DEVICE WITH A BASE BODY

(75) Inventors: Alfred Stett, Reutlingen (DE); Wilfried Nisch, Tuebingen (DE)

Correspondence Address: KLARQUIST SPARKMAN, LLP, 121 SW SALMON STREET, SUITE 1600, PORTLAND, OR, 97204, US

(73) Assignee: NMI Naturwissenschaftliches und Medizinsches Institut an der Universitaet Tuebingen

(21) Appl. No.: 12/421,465

(22) Filed: Apr. 9, 2009

Related U.S. Application Data
(63) Continuation of application No. PCT/EP2007/008435, filed on Sep. 27, 2007.

(30) Foreign Application Priority Data
Oct. 10, 2006 (DE) 10 2006 048 819.9

(51) Int. Cl. A61N 1/36; A61N 1/05

(52) U.S. Cl. 607/54; 607/116

(57) ABSTRACT

On a device 10 with a base body 32 at least one electrode 17 is arranged which serves to exchange electrical or chemical signals with surrounding tissue 34, the electrode 17 being covered by a protective layer 33 which is of such a nature that, after contact with the tissue 34, it decomposes in a defined manner and at least to such an extent that the electrode 17 comes into direct contact with the tissue 34.

### CROSS REFERENCE TO RELATED APPLICATIONS

This is a continuation application of International Patent Application PCT/EP2007/008435, filed Sep. 27, 2007, designating the United States and published in English as WO 2008/043439, which claims priority of German patent application DE 10 2006 048 819.9, filed 10. Oct. 2006. The entire contents of these prior applications are incorporated herein by reference.

### BACKGROUND OF THE INVENTION

1. Field of the Invention

The present invention relates to a device with a base body on which base body at least one electrode is arranged, which electrode serves to exchange electrical or chemical signals with surrounding tissue and is covered by a protective layer. The invention further relates to a method for production of a device of this kind.

2. Related Prior Art

A device of this kind is known from EP 0 388 480 A1, for example.

The known device is an implantable porous stimulation electrode on a cardiac pacemaker, in which the electrode surface is provided with a thin coating composed of a hydrophilic polymer, and in which an anti-inflammatory steroid is embedded in the polymer. In this way, in the known cardiac pacemaker, it is possible to avoid the stimulation thresholds increasing initially after the implantation, which is attributable to inflammatory reactions and scarring in the area of the electrodes.

In the known cardiac pacemaker, the steroid diffuses out of the thin protective layer into the adjoining tissue, with the result that possible inflammatory processes are suppressed and the process of incorporation of the electrode into the trabecular network of the heart muscle is supported. The protective layer is chemically and thermally stable and has proven to be tissue-compatible and biocompatible, such that, if the implant is left for a long period of time in the body, the protective layer ensures a permanent covering of the porous surface of the electrode and protects it from contamination, whereby a loss of capacitance of the porous electrode surface does not occur.

The device mentioned at the outset, however, is to be understood not only in the form of cardiac pacemakers but also, for example, of cochlear implants, which likewise represent an electrically active implant. In addition, the device mentioned at the outset is also understood as meaning electronic micro-implants for stimulation of retinas, in which sight has been lost, and of various regions of the brain, for example epiretinal or subretinal retinal implants, implants for the visual or auditory cortex, or implants for the brain stem. This list is not intended to be exhaustive.

The implants mentioned are used to measure or stimulate neuronal activity and, in the case of control systems, both functions can be performed on the known devices.

However, the device mentioned at the outset is not to be understood only in the form of electrical implants, but also in the form of devices which can be used ex vivo and with which measurements or stimulation experiments can be carried out on cell aggregates, for example, as is the case in dose-finding studies for pharmacological substances or in toxicity measurements for determining maximum workplace concentrations, and in which cultured tissue is used instead of living test animals.

All the devices described hitherto require a stable, durable and functional coupling of the electronic system to the biological system. This is usually achieved by the closest possible mechanical coupling of the electrodes to the surrounding tissue, the electrode surface often being provided with a porous structure having the largest possible internal surface area, such that they have the greatest possible charge transfer capacitance via the Helmholtz double layer at the interface between electrode and the electrolyte in the tissue.

However, in implants with a high electrode density, for example in retinal implants, the surface of the individual electrodes is so small that, even with a porous structure, the charge transfer capacitance is not always sufficient, and instead electrochemical effects on the electrode occur that increase the stimulation thresholds and can even have a cytotoxic or inflammatory action.

The close mechanical coupling to the target cells that are to be stimulated is also limited in planar micro-electrodes, which cannot penetrate into the adjoining tissue. Especially when the electrodes are still isolated from the target cells by non-excitable cell layers, this can considerably increase the stimulation thresholds. This problem arises in particular in retinal implants since, for example, the lost photoreceptors or possible glial scars obstruct the close mechanical contact between the electrodes and the neurons that are to be stimulated.

To bring the electrodes closer to the cells that are to be stimulated and are located within the tissue volume, the prior art therefore proposes needle electrodes that are intended to penetrate into the tissue. In this way, the stimulus strengths needed to reach the required stimulation thresholds are reduced and the local resolution enhanced.

R. A. Normann: “APPLICATIONS OF PENETRATING MICRO-ELECTRODE ARRAYS IN NERVOUS SYSTEM DISORDERS”, in Review of North American Research on Brain Computer Interfaces, WTEC Workshop (2006), pages 66-69, discloses, in this connection the use of penetrating electrodes for establishing particularly selective connections to small groups of nerve cells within tissues.

D. Palanker et al.: “DESIGN OF A HIGH-RESOLUTION OPTOELECTRONIC RETINAL PROSTHESIS”, in J. Neural Eng. (2005) 2(1), pages 105-120, disclose the use of penetrating electrodes in subretinal implants. The electrodes used have a diameter in the range of 10 μm, and a height above the base body in the range of 70 μm. The authors were able to show that, after implantation of an array of such needle electrodes into the subretinal space of a rat, independent migration of the cells into the interstices between the electrodes was observed.

As regards needle electrodes, both when used on implants and also in connection with devices to be used ex vivo, it is of particular importance that the needle tips penetrating into the tissue do not damage any cells within the tissue mass.

W. Shain et al.: “IT'S ALL IN THE SEEING: A BRIEF REVIEW OF THE STUDY OF BRAIN RESPONSES TO INSERTED NEURAL PROSTHETIC DEVICES”, in Review of North American Research on Brain Computer Interfaces, WTEC Workshop (2006), pages 96-98, in this connection draw attention to the fact that the most important factor is the speed with which the needles are inserted into the tissue.

P. J. Rousche and R. A. Normann: “A METHOD FOR PNEUMATICALLY INSERTING AN ARRAY OF PENETRATING ELECTRODES INTO CORTICAL TISSUE”, in Annals of Biomedical Engineering (1992), 20, pages 413-422, disclose an array of 100 needle electrodes designed to penetrate into the tissue. After simple pressing-in of the electrode array proved unsuccessful, the array was inserted into the tissue by means of pneumatic acceleration at a speed of between 1 and 11 m/s. The authors report that a speed of at least 8.3 m/s was required for safely inserting all 100 electrodes within the array to a depth of 1.5 mm into cortical tissue.

On the other hand, H. Thielecke et al.: “GENTLE CELL HANDLING WITH AN ULTRA-SLOW INSTRUMENT: CREEP-MANIPULATION OF CELLS”, in Microsyst Technol (2005), 11, pages 1230-1241, report that it is possible for micro-electrodes with tip diameters of a few μm to be inserted without cell damage into a three-dimensional cell aggregate if the movement of the electrodes only takes place slowly. If this speed is in the range of the speed of migration of the cells, the cells are able to give way to the advancing electrodes, such that they are not destroyed. The authors report that speeds of advance in the range of a few nm/s have proven successful.

B. Niggemann et al.: “THE MINIMAL INVASIVE RETINAL IMPLANT (miRI) PROJECT: FIRST SERIES OF IMPLANTATION WITH LONG-TERM FOLLOW-UP IN NONHUMAN PRIMATES”, in Invest Opthalmol Vis Sci (2006), 47: E-Abstract, page 1031, disclose an application of this slow tissue penetration in which electrodes are advanced into the subretinal space, without the latter having to be opened surgically. To do this, electrodes are placed externally onto the sclera, and a continuous and light pressure exerted by the tissue that covers the electrodes has the effect that, within weeks or months, the electrodes penetrate into the sclera and migrate slowly forwards into the subretinal space.

As has already been mentioned at the outset, the implantation of electrodes is often followed by scarring at the interface between electrode and tissue, as a result of which the electrical properties of this interface layer alter in a disadvantageous manner. On the one hand, the scar layer increases the electrical resistance of the contact and, on the other hand, this has the result that the distance between the electrode surface and the target cells increases. Both of these phenomena have the effect that the stimulation efficiency decreases, and this has to be compensated by increased charge transfer.

### SUMMARY OF THE INVENTION

In view of the above, it is an object of the present invention to develop the device mentioned at the outset in such a way that, with different shapes and dimensions of the electrodes, it can be brought gently into contact with the tissue in such a way that the electrode tips are brought close to the target cells within the tissue, where they can be placed in a stable position.

According to the invention, in the device mentioned at the outset, this and other objects are achieved by the fact that the protective layer is of such a nature that, after contact with the tissue, it dissolves or breaks up in a defined manner and at least to such an extent that the electrode comes into direct contact with the tissue.

The object underlying the invention is achieved in full in this way.

The inventors of the present application have indeed found that not only does a self decomposing protective layer, upon implantation, or upon any other use of the device, protect the electrodes against damage associated with the required manipulations, but also that the tissue with which the device is brought into contact does not suffer any damage, since the possibly sharp tips of the electrodes are covered by the protective later during their application.

In this way, therefore, it is possible for the novel device to be implanted in tissue, for example, without any danger of the electrodes or the tissue being damaged.

The device according to the invention can therefore also be introduced into the interior of tissue volumes that are difficult to access, without these being mechanically damaged during the generally complex surgical implantation.

A protective layer that “breaks up or dissolves in a defined manner” is to be understood as a protective layer that independently decomposes in a targeted way, i.e. intentionally, or in a predetermined manner, after contact with the tissue. In other words, the protective layer is continuously degraded within a period stretching between the time when the contact between tissue and protective layer is established and the time when the device is set to use. The degradation of the protective layer can be triggered by chemical, biological or physical processes. It is only after the device has been placed at the intended site that the protective layer begins to break up and the electrodes are freed and gradually come into contact with the tissue.

It is preferable if the electrode is a needle electrode and is preferably arranged in an array of needle electrodes.

The inventors of the present application have indeed also found that many problems surrounding the implantation of such devices can be avoided if the needle electrodes arranged in an array are embedded in a protective layer that decomposes after implantation. According to the invention, the layer is degraded and/or absorbed after implantation, such that the needle tips come into contact with the tissue. The material from which the protective layer is made is chosen or modified such that the rate of degradation is well defined. With slow degradation of the protective layer, the tips of the needle electrodes are freed gradually by the protective layer and come into contact with the cells which, in line with their speed of migration, give way to the tips. The needle electrodes thus penetrate slowly into the tissue without damaging it. The penetration is assisted by the fact that the tissue into which the device has been implanted exerts a pressure on the array, such that a force arises in the direction of the needle axes.

In this way, it is now also possible to advance needle electrodes into deeper-lying tissue layers or into tissue layers that are difficult to access, and this without the danger of mechanical damage to the electrodes or to the tissue.

In principle, implants can be advanced into the desired tissue layers only from a freely accessible surface, in which case electrodes projecting from the base body perpendicular to the direction of advance may be damaged by the mechanical advance while, on the other hand, there is the danger of these electrodes damaging the tissue along the path of advance through the tissue.

Moreover, for the penetration of the needle electrodes into the tissue, mechanical pressures are needed, which have to act on the electrodes in the direction of advance. This means, however, that in the prior art needle electrodes could only be advanced into cell layers that were accessible from the outside using mechanical aids; see H. G. Sachs et al.: “TRANSSCLERAL IMPLANTATION AND NEUROPHYSIOLOGICAL TESTING OF SUBRETINAL POLYIMIDE FILM ELECTRODES IN THE DOMESTIC PIG IN VISUAL PROSTHESIS DEVELOPMENT”, in J. Neural Eng. (2005), 2 (1), pages 57-64, showing common operating techniques for retinal implants.

Moreover, controlled speeds of penetration can be established only with difficulty if the electrodes, after intracortical localization, are intended to penetrate slowly into the desired cell volume, in which case surgical interventions lasting for days were necessary in some circumstances. See W. Shain et al., loc. cit.

In the context of the present application, a needle electrode is understood not only as meaning long cylindrical electrodes that possibly taper towards their tip, but also any other type of electrode protruding from the base body. The electrodes can be made completely or partially of conductive materials, in which case, for example, it is also possible for only the tip of the electrode to be made conductive.

In the context of the present invention, a base body is understood for example as a flexible film on which, in addition to the electrodes, various other electronic components are also arranged. However, the base body can also be made of any other material, including stiff material. Finally, it is also not necessary for other electronic components to be arranged on the base body in addition to the electrodes; in other words, the base body can merely comprise the electrode array, in which case the electrode array is connected to stimulation and/or measurement electronics by way of multi-core feed lines, for example a flat ribbon cable.

At their tip, the electrodes preferably have dimensions of the order of magnitude of cellular structures such as axons, dendrites or cell bodies, their diameter preferably being in the range of 1 to 10 μm.

To be able to drive the needle electrodes sufficiently far into the tissue, the electrodes are preferably needle-shaped and have lengths in the range of up to several 10 μm.

In one embodiment, it is preferable if the electrode is a hollow electrode.

In the context of the present invention, a hollow electrode is understood as an electrode with a channel which passes through it lengthwise and via which chemical signals can be exchanged with the surrounding tissue. This is of advantage, for example, when the stimulation of the tissue in contact with the electrodes is intended to take place by way of chemical substances, or when chemical substances are intended to be removed locally from the tissue.

According to another object, the protective layer comprises biodegradable and/or bioabsorbable materials with a defined rate of degradation.

The advantage of this measure is that materials of this kind have a slow rate of degradation, such that the slow degradation of the protective layer permits the controlled and slow penetration of the electrodes into the tissue, the contact pressure being applied by the tissue that lies as it were on the other side of the device. In other words, the speed at which the electrodes, preferably the needles, penetrate into the tissue is defined by the rate at which the protective layer is degraded.

Biodegradable and bioabsorbable materials are known per se and are widely used in the production and use of biomedical implants. They are distinguished by their biocompatibility and by their natural ability to decompose in the tissue over the course of time. They are used in orthopaedics, wound treatment or drug delivery. The most common materials are polylactic acid (PLA), polyglycolic acid (PGA) and their copolymers, and polycaprolactone (PCL). The protective layer can also contain gelatin or be composed substantially of gelatin.

The use of these polyesters in the protective layer is particularly preferable since they degrade especially easily by simple hydrolysis, with the hydrolysis products being absorbed by normal metabolic processes, and they additionally allow the rate of degradation to be set in a targeted or desired manner. Factors defining the rate of degradation are, in addition to the exact molecular structure, also the ratio of the copolymers to one another, the molecular weight and, if appropriate, also the production method itself. The rates of degradation of these polymers lie in the range of 1 to 24 months.

An overview of the materials that can be used in the context of the present invention is provided by P. A. Gunatillake and R. Adhikari: “BIODEGRADABLE SYNTHETIC POLYMERS FOR TISSUE ENGINEERING”, in European Cells and Materials (2003), 5, pages 1-16. Depending on the type of use of the novel device and on the desired speed of penetration of the electrodes into the corresponding tissue, the known materials can be combined such that they have the corresponding rate of degradation. Against this background, the disclosure of the above publication by Gunatillake and Adhikari loc. cit. is hereby incorporated by reference into the present application.

Bioabsorbable materials for biomedical applications and biodegradable polyesters are also widely described in the prior art. In contrast to pure polymers, implants made of biodegradable polyesters (poly(L-lactide) and poly(D,L-lactide)) and of amorphous, carbonate-containing calcium phosphate or calcium carbonate, respectively, have the advantage that they do not release acid products upon degradation and instead have a physiological pH value, since the resulting acids are buffered by the inorganic filler; see, for example, C. Schiller: “NEUE MATERIALIEN IM KOPF: SCHÄDELIMPLANTAT LöST SICH VON INNEN HER AUF” in www.uni-protokolle.de/nachrichten/id/25555/.

Moreover, bioabsorbable layers of metal or of metal alloys are also highly suitable, e.g. magnesium and magnesium alloy (see www.unics.uni-hannover.de/analytik/Forschung/Bioresorbierbare%20Implantate.pdf).

In this context, DE 100 28 522 A1 discloses a biodegradable neuro-electrode which is provided with a mechanical support element made of biodegradable material, in order to allow the implant, which is not itself mechanically stable, to be handled during the implantation.

By way of comparison, U.S. Pat. No. 6,792,315 B2 discloses an electrode arrangement which can be implanted into the eyelid and which is arranged on a support made of biodegradable material that decomposes after implantation. In this case too, during implantation, the electrode arrangement is stabilized by the support, such that it can be better handled.

Neither in DE 100 28 522 A1 nor in U.S. Pat. No. 6,792,315 B2, does the biodegradable structure cover the electrodes, in each case it performs only a mechanical support function. During implantation, the electrodes in these known devices are not protected against damage and come directly into contact with the tissue during the intervention.

The use of biodegradable and/or bioabsorbable materials as a temporary protective layer as it were on an array of needle electrodes in an implantable device is not hitherto described in the prior art.

As has already been mentioned, the novel device can be used both ex vivo and also in various medical implants, but it is particularly preferably designed as an active retinal implant with a multiplicity of image cells converting incident light into electrical signals, which are delivered via the electrodes to surrounding tissue.

A retinal implant of this kind is known, for example, from WO 2005/000395 A1, the disclosure of which is hereby incorporated by reference into the present application. The retinal implant is supplied wirelessly with electrical energy via irradiated IR light or via inductively coupled-in HF energy, and this external energy can include information concerning the control of the implant.

However, since wireless retinal implants of this kind for use on humans are not available with sufficient quality, not only epiretinal but also subretinal implants are currently proposed in which the required external energy is supplied by wire.

Thus, Gekeler et al.: “COMPOUND SUBRETINAL PROSTHESES WITH EXTRA-OCULAR PARTS DESIGNED FOR HUMAN TRIALS: SUCCESSFUL LONG-TERM IMPLANTATION IN PIGS”, Graefe's Arch Clin Exp Opthalmol (28 Apr. 2006) (e-publication ahead of print) disclose a subretinal implant in which the external energy and the required control signals are guided by wire to the chip implanted in the eye. The disclosure of this publication too is hereby incorporated by reference into the present application.

Sachs et al., loc. cit., also disclose methods for subretinal implantation in which film electrodes are pushed into the retina, i.e. between pigment epithelium and neuronal retina. Here, particular care must be taken to ensure that the implant is not damaged during insertion and that the retina is not damaged by the mechanical pushing in between the cell layers.

With the design of the retinal implant according to the invention, it is now possible to provide such implants with an array of protruding needle electrodes, without encountering the stated problems during implantation. After the implantation, the protective layer decomposes such that the layers of the retina can settle gradually on the needle tips, and, as the absorbable material continues to decompose, the needles penetrate further into the tissue, without cells being damaged thereby.

According to a further object, the protective layer incorporates biologically active substances that are released when the protective layer decomposes.

This measure has the advantage that, as the protective layer degrades, active substances can at the same time be released into the surrounding tissue which, for example, prevent scar formation or have an anti-inflammatory action.

It is particularly preferable if the active substance is an anti-inflammatory steroid, as is known, for example, from EP 0 388 480 A1 mentioned at the outset. The steroid used is preferably dexamethasone and/or cortisone.

According to still a further object, the electrode comprises a base electrode from which a multiplicity of needle electrodes project.

This measure has the advantage that a mechanically closer coupling to the target cells to be stimulated is achieved than in the case of the base electrode on its own. Moreover, a lower stimulation threshold is needed, since the surface of the base electrode extends as it were to the target cells and the dendrites present on nerve cells in the biological tissue, the electrodes also having a similar order of magnitude.

Therefore, the electrode is as it were a three-dimensional nanoelectrode in which the multiplicity of needle electrodes, which as it were represent a nanoscale part of the electrode, can penetrate as gently as possible into the adjoining cell layer.

Such nanoelectrode structures composed of a planar base electrode with a multiplicity of projecting needle electrodes can be produced, for example in a manner known per se in other contexts, with an electron beam writer by electron beam exposure of suitable masks and subsequent plasma etching or can be directly etched with an FIB (focussed ion beam) appliance or deposited reactively.

On the other hand, it is also possible to design the nanoelectrode structure in UV-curable polymer which is applied to a substrate surface in a spin-coating technique. A nanostamp is pressed into the polymer, after which the polymer is cured and finally removed from the mould. The nanostructure thus formed in the polymer by the nanoprinting technique is then coated with the electrode material by reactive sputtering with TiN, Ir or IrO. After application of the steroid, the structure can then be coated with the biodegradable material.

The nanostamp therefore only has to be produced once, for example by electron beam writing, and it is then possible, in principle, to produce any desired number of nanostructures with the nanostamp.

It is also generally preferable if the electrode is made from a flexible material.

This measure has the advantage that, because of the protective layer initially provided on the base body and covering the electrodes, it is also possible to use flexible electrodes for implants or ex vivo devices without the danger of the flexible electrodes being damaged during handling of the device. Flexible electrodes also have the advantage that, as the protective layer decomposes, they are able to give way to certain structures in the tissue, such that they permit gentle penetration.

The flexibility of the material can be achieved through the properties of the material and also through the dimensions of the material. Particularly thin needle electrodes, for example, then also have a certain flexibility if they are made of metal.

It is generally also preferable if at least one planar electrode is provided on the base body.

This planar electrode can be used in a manner known per se to ground the device to the surrounding tissue. It is possible to arrange the needle-shaped electrodes and the planar electrode on different sides of the base body, and it is also possible to arrange both types of electrodes on the same side of the base body.

Finally, it is also possible to provide needle-shaped electrodes and/or planar electrodes on both sides of the base body.

These measures together have the advantage that the novel device can be implanted at any desired location in a tissue, the delivery of stimulation signals or the measurement of states of excitation can take place on one or both sides of the device, and the grounding to the surrounding tissue can also be suitably provided.

In view of the above observations, the present invention also relates to a method for protecting an electrode arrangement which is provided on a base body and which serves to exchange electrical or chemical signals with surrounding tissue and is embedded in a protective layer, wherein the protective layer is of such a nature that, after contact with the tissue, it decomposes in a defined manner and at least to such an extent that the electrode arrangement comes into direct contact with the tissue. The method is preferably carried out on the novel device described above.

As has already been mentioned, this measure has the advantage that the electrode arrangement is protected when being handled before its use and during its use, in particular during the implantation itself. The degradation of the protective layer can be triggered by chemical, biological or physical processes.

Finally, the present invention also relates to a method for establishing contact between a tissue and an electrode arrangement which is provided on a base body of a device and which serves to exchange electrical or chemical signals with the surrounding tissue and is embedded in a protective layer, wherein the protective layer, after contact with the tissue, is decomposed in a controlled manner, such that the tips of the electrode arrangement come into contact with the tissue and gradually penetrate into the latter. The method is preferably carried out on the novel device described above.

Further advantages will become clear from the description and from the attached drawing.

It will be appreciated that the aforementioned features, and the features still to be explained below, can be used not only in the respectively cited combination but also in other combinations or singly, without departing from the scope of the present invention.

### BRIEF DESCRIPTION OF THE DRAWINGS

An embodiment of the invention is explained in more detail in the following description and is depicted in the drawing, in which:

FIG. 1 shows a schematic view of an implantable device, in this case a retinal implant, in a representation not true to scale;

FIG. 2 shows a schematic view of a human eye into which the retinal implant according to FIG. 1 is fitted, again in a representation not true to scale;

FIG. 3 shows a schematic view of the retinal implant from FIG. 1;

FIG. 4 shows a schematic view of the implantation of the retinal implant from FIG. 3 into surrounding tissue; and

FIG. 5 shows a schematic view of an electrode array being brought into contact with tissue ex vivo.

### DETAILED DESCRIPTION OF A PREFERRED EMBODIMENT

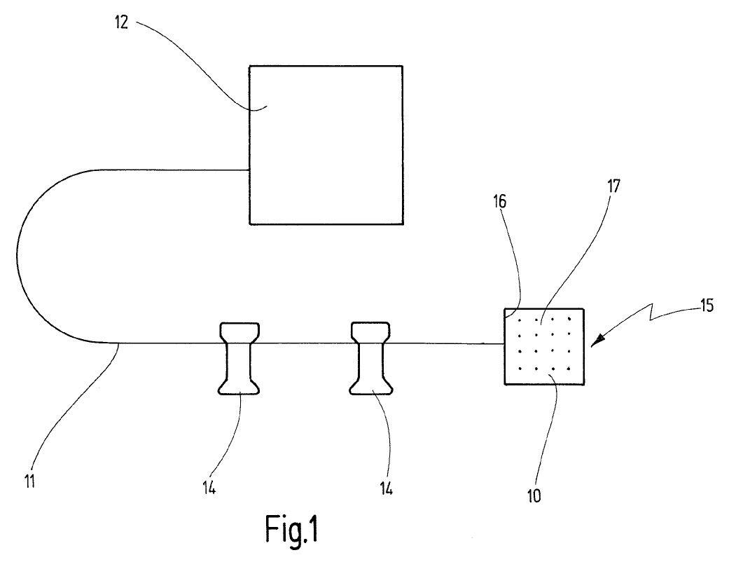

An example of the novel device is shown schematically in FIG. 1 in the form of an implantable device 10, the dimensions of which are not represented true to scale. The device 10 is connected via a cable 11 to a supply unit 12, which supplies the device 10 with electrical energy and with control signals. Securing patches 14 are provided on the cable 11 and can be used to secure the cable on the body of the person in whom the implant 10 is fitted.

The device 10 can be any desired type of implant that excites electrically excitable cells. In the case shown, it is an active retinal implant 15 which, as its base body, has a film 16 on which electrodes 17 for delivering stimulation signals to excitable cells are arranged.

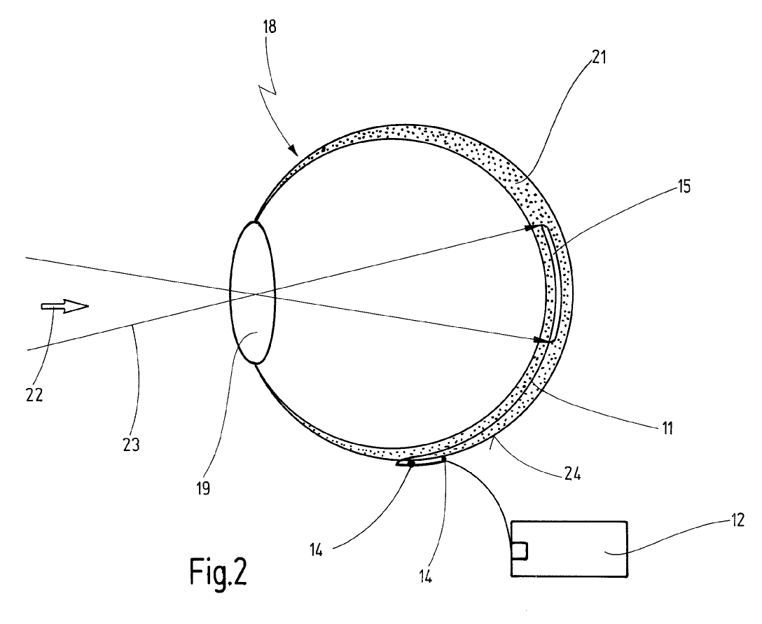

The retinal implant 15 from FIG. 1 is designed to be implanted into a human eye 18, which is depicted very schematically in FIG. 2. To keep matters simple, the figure shows only the lens 19, and the retina 21 into which the implant 15 is fitted. The implant 15 is preferably fitted in what is called the subretinal space, which is formed between the pigment epithelium and the photoreceptor layer. If the photoreceptor layer is degenerated or absent, the subretinal space forms between the pigment epithelium and the layer of bipolar and horizontal cells. The retinal implant 15 is placed in such a way that stimulation signals can be delivered to cells in the retina 21 via the electrodes 17 shown in FIG. 1.

Visible light, which is indicated by an arrow 22 and whose beam path can be seen at 23, is conveyed through the lens 19 onto the implant 15, where the visible light 22 is converted into electrical signals, which are converted into stimulation signals.

It will be noted that the cable 11 is routed laterally out of the eye and is secured there on the outside of the sclera 24 by the securing patches 14, after which the cable leads onwards to the external supply unit 12.

The supply unit 12 is then secured, in a manner not shown, outside the eye, for example on the patient's skull. Electrical energy is sent to the implant 10 via the supply unit 12, and at the same time control signals can also be transmitted that influence the mode of operation of the implant in the manner described, for example, in the aforementioned WO 2005/000395 A1, the content of which is hereby incorporated by reference into the present application.

It will also be noted that the dimensions of the retinal implant 15 in particular, of the securing patches of the external supply device 12 in FIGS. 1 and 2 are not true to scale, nor are these shown in the correct relationship to one another in terms of their size.

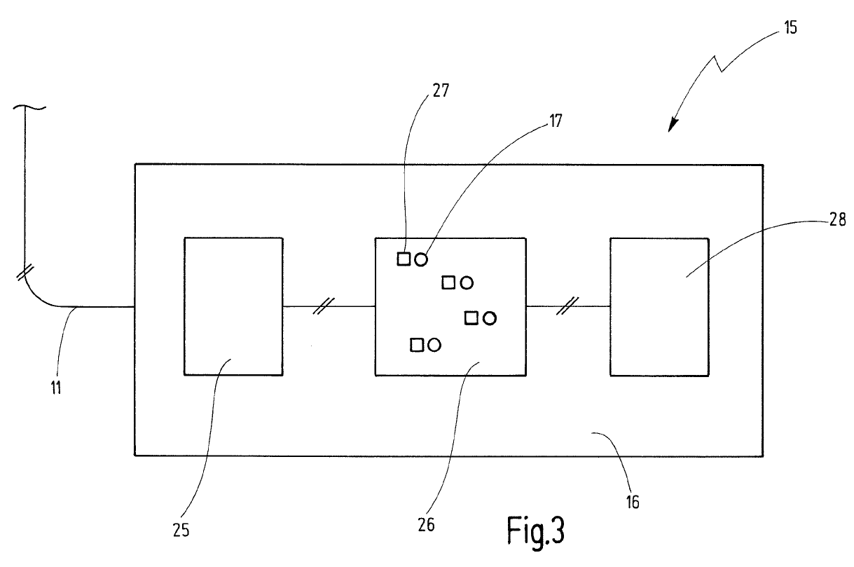

FIG. 3 is a schematic view of the configuration of the active retinal implant 15 from FIG. 1. On the film 16, there is in the first instance an input stage 25, which is supplied with external energy from outside via the cable 11. The input stage 25 is connected to a sensor unit 26, which in this case has a multiplicity of image cells 27 converting incident visible light into electrical signals, which are then delivered to nerve cells of the retina via the electrodes 17 indicated alongside the respective image cells.

The useful signals generated by the image cells 27 are processed in an output stage 28, which generates the corresponding stimulation signals, and these are then fed back to the sensor unit 26 and to the electrodes 17.

In this connection, it will be noted that FIG. 3 is only a schematic representation of the retinal implant 15 showing the logic layout; the actual geometric arrangement of the individual components may, for example, entail each image cell 27 having an output stage in its immediate proximity.

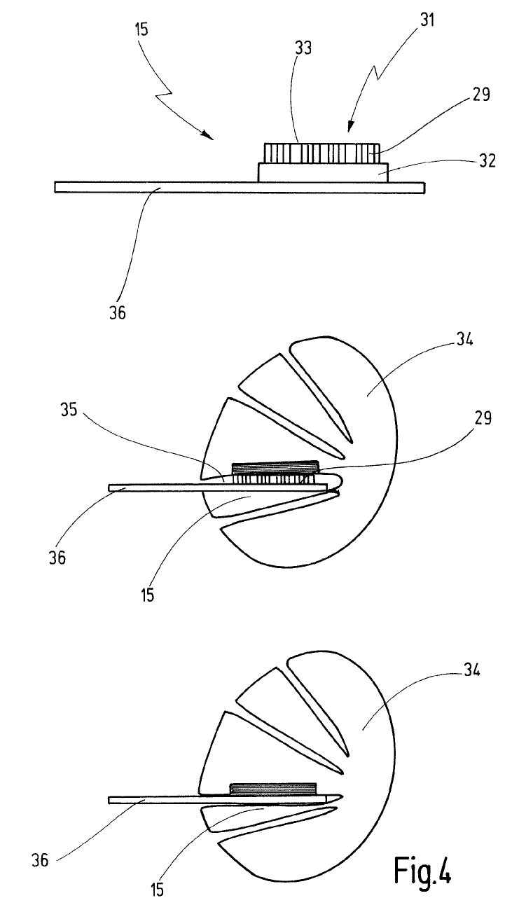

The electrodes 17 can be designed as needle electrodes 29, for example, and can be arranged in a separate array 31 on a base body 32, as is shown now at the top of FIG. 4. The needle electrodes 29 have, for example, a diameter of 10 Mm and a height above the base body 32 of 70 Mm, and they taper upwards.

The electrodes 17 are in this case covered by a protective layer 33 made of a biodegradable and/or bioabsorbable material with a defined rate of degradation. Biologically active substances are incorporated into the protective layer 33 and are released when the protective layer 33 decomposes.

The biologically active substances have an anti-inflammatory action and also promote or inhibit cell growth. In many cases, a steroid such as cortisone and/or dexamethasone is embedded into the protective layer 33.

This base body 32 is now implanted in tissue, indicated by 34, for which purpose it is inserted into an incision 35.

The base body 32 is held on a support 36 via which the retinal implant 15 is now pushed into the incision 35, as is shown in the middle picture in FIG. 4.

The incision 35 now presses onto the implant 15, as a result of which the protective layer 33 comes into contact with the tissue 34 and gradually degrades. As the protective layer 33 degrades, the needle electrodes 29 penetrate into the tissue 34 until, finally, said needle electrodes 29 are received completely within the tissue 34, as is shown at the bottom of FIG. 4.

While the implant 15 is being pushed into the tissue 34, the electrodes 17 are thus protected by the protective layer 33 and, at the same time, structures of the tissue 34 cannot be damaged during this pushing in.

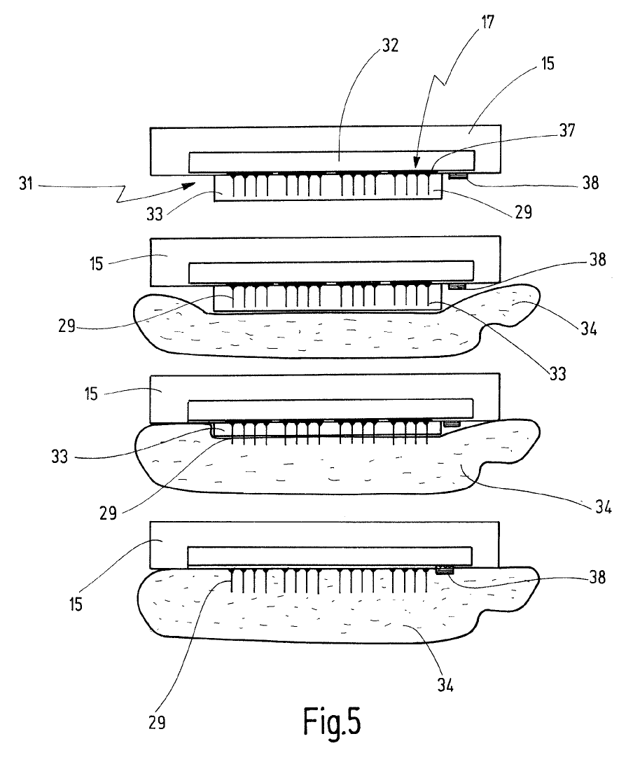

FIG. 5 shows, once again schematically, the penetration of the needle electrodes 29 into a tissue 34, which lies ex vivo. Here too, the retinal implant 15 can be used, for example when it is being tested ex vivo.

The implant 15 is shown in a schematic side view at the top of FIG. 5, where it can be seen that each electrode 17 in the array 31 of electrodes 17 comprises a respective base electrode 37 from which several needle electrodes 29 project. In this way, each electrode 17 delivering the signal of an image cell to the retina is provided with several needle electrodes 29, such that there is good mechanical and electrical coupling of the electrode 17 to the tissue 34.

The schematic depiction of the implant in FIG. 5 also shows a planar electrode 38, which serves for grounding the implant 15 to the tissue 34, as is known per se.

As has already been described in detail in the introductory part of the description, the electrode array 31 from FIG. 5, with the base electrodes 37 and the projecting needle electrodes 29, is produced either by electron beam writing with subsequent plasma etching or by a nanoimprint technique in which a nanostructure is incorporated into a UV-curable polymer and the electrode material of TiN, Ir or IrO is then applied by reactive sputtering onto the nanostructure thereby generated. The steroid is then applied, and this is followed by application of the biodegradable protective layer 33.

The retinal implant is pressed onto the tissue, for example by its own weight or by a force exerted from outside, that is to say from above in FIG. 5, such that the needle electrodes 29 slowly advance into the tissue 34 when the protective layer 33 is degraded.

The protective layer 33 can be caused to degrade solely by contact with the tissue 34, but it is also possible to trigger the degradation by chemical, biological or physical processes.

We claim:

1. A device having a base body, at least one electrode being arranged on said base body, wherein said electrode is arranged for exchanging electrical or chemical signals with surrounding tissue, said at least one electrode is covered by a protective layer,
wherein the protective layer is made from material of such a nature that, after contact with the tissue, decomposes in a defined manner and at least to such an extent that said at least one electrode comes into direct contact with said tissue.

2. The device of claim 1, wherein the electrode is a needle electrode.

3. The device of claim 2, wherein an array of needle electrodes is arranged on said base body.

4. The device of claim 1, wherein the electrode is a hollow electrode.

5. The device of claim 1, wherein the protective layer comprises a material selected from the group consisting of biodegradable and bioabsorbable materials having a defined rate of degradation.

6. The device of claim 5, wherein the protective layer (33) is selected from the group consisting of polyglycolic acid, L-polylactic acid, D,L-polylactic acid, polycaprolactone, copolymers thereof, gelatin, biodegradable metals, metal alloys, magnesium and magnesium alloys.

7. The device of claim 1, wherein the protective layer incorporates biologically active substances that are released when the protective layer decomposes.

8. The device of claim 7, wherein the biologically active substance is selected from the group consisting of anti-inflammatory substances, cell growth promoting substances, cell growth inhibiting substances, steroids, cortisone and dexamethasone.

9. The device of claim 1, comprising a multiplicity of image cells that convert incident light into electrical signals, which electrical signals are delivered via the electrodes to surrounding tissue.

10. The device of claim 1, wherein the electrode comprises a base electrode and a multiplicity of needle electrodes projecting from said base electrode.

11. The device of claim 1, wherein the electrode is made from a flexible material.

12. The device of claim 1, wherein at least one planar electrode is provided on the base body

13. An active retinal implant comprising a multiplicity of image cells that convert incident light into electrical signals, a base body, a multiplicity of electrodes being arranged on said base body, wherein said electrodes are arranged for delivering said electrical signals to tissue surrounding said retinal implant when in use, said multiplicity of electrodes being covered by a protective layer, said protective layer being made from such a material that, after contact with the tissue, decomposes in a defined manner and at least to such an extent that said electrodes come into direct contact with said tissue.

14. The retinal implant of claim 13, wherein the protective layer comprises a material selected from the group consisting of biodegradable and bioabsorbable materials having a defined rate of degradation.

15. The retinal implant of claim 14, wherein the protective layer (33) is selected from the group consisting of polyglycolic acid, L-polylactic acid, D,L-polylactic acid, polycaprolactone, copolymers thereof, gelatin, biodegradable metals, metal alloys, magnesium and magnesium alloys.

16. The retinal implant of claim 13, wherein the protective layer incorporates biologically active substances that are released when the protective layer decomposes.

17. The retinal implant of claim 16, wherein the biologically active substance is selected from the group consisting of anti-inflammatory substances, cell growth promoting substances, cell growth inhibiting substances, steroids, cortisone and dexamethasone.

18. A method for protecting, during implantation into a tissue, an electrode arrangement which is provided on a base body of a device and which is arranged to exchange electrical or chemical signals with surrounding tissue, said electrode arrangement being embedded in a protective layer, wherein the protective layer is made of such a material that, after contact with the tissue, decomposes in a defined manner and at least to such an extent that the electrode arrangement comes into direct contact with the tissue.

19. The method of claim 18, wherein the protective layer comprises a material selected from the group consisting of biodegradable and bioabsorbable materials having a defined rate of degradation.

20. The method of claim 19, wherein the protective layer (33) is selected from the group consisting of polyglycolic acid, L-polylactic acid, D,L-polylactic acid, polycaprolactone, copolymers thereof, gelatin, biodegradable metals, metal alloys, magnesium and magnesium alloys.

21. A method for establishing contact between a tissue and an electrode arrangement which is provided on a base body of a device, said electrodes having tips and being arranged for exchanging electrical or chemical signals with the surrounding tissue, said electrode arrangement being embedded in a protective layer, wherein the protective layer is made of such a material that, after contact with the tissue, is decomposed in a controlled manner, such that the tips of the electrode arrangement come into contact with the tissue and gradually penetrate into the latter.

22. The method of claim 21, wherein the protective layer comprises a material selected from the group consisting of biodegradable and bioabsorbable materials having a defined rate of degradation.

23. The method of claim 22, wherein the protective layer (33) is selected from the group consisting of polyglycolic acid, L-polylactic acid, D,L-polylactic acid, polycaprolactone, copolymers thereof, gelatin, biodegradable metals, metal alloys, magnesium and magnesium alloys.
